(19) United States
(12) Patent Application Publication
(10) Pub. No.: US 2018/0198272 A1
(43) Pub. Date: Jul. 12, 2018

(54) Zener Overvoltage Protection (OVP) with a Thermal Trigger

(71) Applicant: QUALCOMM Incorporated, San Diego, CA (US)

(72) Inventor: Mark D. White, II, Solana Beach, CA (US)

(21) Appl. No.: 15/404,510

(22) Filed: Jan. 12, 2017

(51) Int. Cl. H02H 7/125; H02J 50/10; A61N 1/378

(52) U.S. Cl. CPC H02H 7/1252; A61N 1/3787; H02J 50/10

(57) ABSTRACT

The present disclosure describes aspects of Zener overvoltage protection (OVP) with a thermal trigger for an implant. In some aspects, an apparatus comprises a rectifier disposed in a rectification circuit and configured to rectify alternating current (AC) power received at the rectification circuit to direct current (DC) power. The apparatus also includes a Zener diode and thermistor. The Zener diode is disposed in the rectification circuit between the rectifier and an output terminal, and is configured to clamp the DC power at a predefined voltage. The clamped DC power output at the output terminal is usable by the implant. The thermistor and Zener diode are disposed in temperature-measuring proximity. This enables the thermistor to measure a temperature of the Zener diode, which correlates to voltage at the Zener diode due to clamping. When the measured temperature exceeds a threshold temperature indicative of an OVP condition, remedial OVP actions are initiated.

### FIELD OF THE DISCLOSURE

This disclosure relates generally to protecting components of a wireless power receiver in a body. More particularly, the disclosure relates to providing overvoltage protection for a power receiving unit using a Zener diode with a thermal trigger.

### BACKGROUND

This description of related art is provided for the purpose of generally presenting a context for the disclosure that follows. Unless indicated otherwise herein, concepts described in this section are not prior art to this disclosure and are not admitted to be prior art by inclusion herein.

Biomedical implants are becoming more common for treatment of disease and medical conditions in humans as well as in animals. These implants can be inserted into a host's body for a variety of purposes, such as to release metered doses of medication, stimulate bodily tissue (e.g., nerves), monitor specific biochemical conditions, and so on. Oftentimes, such implants require electrical energy in order to operate—they need a power source, which typically takes the form of a chemical battery. Although implants are expected to be operative for several years (or a host's lifetime) without replacement, the chemical batteries used to power them may not be capable of operating that long. Thus, to keep these implants operating as designed, their batteries may need to be changed. Changing chemical batteries that are implanted can be difficult, however, and doing so can pose a significant risk to the host. Accordingly, conventional techniques for powering implants can put a host's life at risk

### SUMMARY

In some aspects of Zener overvoltage protection (OVP) with a thermal trigger for an implant, an apparatus comprises a rectifier that is disposed in a rectification circuit and is configured to rectify alternating current (AC) power received at the rectification circuit to direct current (DC) power. The apparatus also includes a Zener diode and a thermistor. The Zener diode is disposed in the rectification circuit between the rectifier and an output terminal of the rectification circuit, and is configured to clamp the DC power at a predefined voltage. The clamped DC power that is output at the output terminal is usable by the implant to carry out its corresponding operations. The thermistor and the Zener diode are disposed in temperature-measuring proximity. This enables the thermistor to measure a temperature of the Zener diode, which correlates to voltage at the Zener diode due to clamping. When the measured temperature exceeds a threshold temperature indicative of an OVP condition, remedial OVP actions are initiated.

Some aspects of Zener OVP with a thermal trigger also involve a method in which AC power is received at a power receiving unit's rectification circuit. The AC power is generated based on wireless power received from a wireless power transmitter. The method also comprises rectifying the AC power to DC power with a rectifier of the rectification circuit. A Zener diode of the rectification circuit clamps the DC power at a predefined voltage, such that the clamped DC power output at an output terminal of the rectification circuit is usable for operation of the implant. Further, the method includes determining that a measured temperature of the Zener diode exceeds a threshold temperature. When the measured temperature exceeds the threshold temperature, the method includes initiating remedial OVP actions.

In other aspects, an apparatus includes biomedical-implant circuitry configured to provide biomedical functionality for a body. The apparatus also includes a power receiving unit that is configured to receive power from a wireless power transmitter and that the biomedical-implant circuitry uses to provide the biomedical functionality. The power receiving unit is also configured to provide overvoltage protection for the biomedical-implant circuitry. In one or more aspects, the power receiving unit includes a rectifier that is disposed in a rectification circuit of the power receiving unit and is configured to rectify AC power received at the rectification circuit to DC power. The power receiving unit also includes a Zener diode to clamp the DC power at a predefined level such that the clamped DC power is usable by the biomedical-implant circuitry to provide its corresponding functionality. Further, the power receiving unit includes a thermistor for measuring a temperature of the Zener diode. The temperature of the Zener diode correlates to a voltage at the Zener diode due to clamping and can be used to trigger overvoltage protection actions.

In aspects, an apparatus for providing Zener OVP with a thermal trigger comprises a rectification means for rectifying AC power received at an input of a rectification circuit to DC power. The apparatus also comprises a voltage-clamping means for clamping the DC power at a predefined voltage. The clamped DC power output from the rectification means is usable by the implant to carry out its corresponding functionality. Further, the apparatus comprises a temperature-measuring means for measuring a temperature of the voltage-clamping means. The measured temperature correlates to voltage stress on the voltage-clamping means and is used to detect an OVP condition.

### BRIEF DESCRIPTION OF DRAWINGS

The details of various aspects are set forth in the accompanying figures and the detailed description that follows. In the figures, the left-most digit of a reference number identifies the figure in which the reference number first appears. The use of the same reference numbers in different instances in the description or the figures indicates like elements:

FIG. 1 illustrates an example environment that includes an implanted electronic device that utilizes Zener overvoltage protection (OVP) with a thermal trigger.

FIG. 2 illustrates an example configuration of a rectification circuit of the power receiving unit shown in FIG. 1.

FIG. 3 illustrates an example of a Zener diode and a thermistor within temperature-measuring proximity for providing Zener OVP with the thermal trigger.

FIG. 4 illustrates an example method for providing Zener OVP with a thermal trigger for an implanted electronic device in a body.

FIG. 5 illustrates an example method for configuring an implanted electronic device to provide Zener OVP with a thermal trigger.

FIG. 6 illustrates a system-on-chip (SoC) having components through which aspects of Zener OVP with a thermal trigger can be implemented.

FIG. 7 illustrates a wireless power transfer system having components through which aspects of Zener OVP with a thermal trigger can be implemented.

### DETAILED DESCRIPTION

Devices implanted in humans and animals are becoming more common, such as biomedical implants capable of treating disease and medical conditions. As used herein, a “host” refers to a respective body (e.g., human or animal) in which an implant is surgically inserted. Biomedical implants can be inserted into a host's body to provide a variety of biomedical functionality, such as to release metered doses of medication, stimulate bodily tissue (e.g., nerves), monitor specific biochemical conditions, and so on. Many implants (biomedical or otherwise) often require electrical energy in order to operate. In other words, these implants need a power source. Often the power source used to power an implant is a chemical battery. Broadly speaking, implants are capable of operating for several years (or a host's entire lifetime) without replacement. The chemical batteries used to power these implants, however, often are not capable of providing power that long. Thus, to keep an implant operating as designed, its battery may need to be surgically changed. The surgical procedures for changing implanted chemical batteries can be invasive and difficult to perform, however. Furthermore, doing so can pose a significant risk to the host. Accordingly, conventional techniques for powering implants can put a host's life at risk.

This disclosure describes aspects of providing Zener diode based overvoltage protection (OVP) with a thermal trigger. The apparatuses and methods described herein prevent overvoltage for components of an implant charged with power received wirelessly. In particular, aspects involve configuring a power receiving unit of an implanted electronic device with a Zener diode and disposing a thermistor so that the Zener diode is within temperature-measuring proximity of the thermistor. In contrast to some OVP techniques that utilize Zener diodes, the aspects described herein incorporate the Zener diode on a direct current (DC) side of a power receiving unit's rectification circuit—rather than on an alternating current (AC) side of the circuit. Incorporating a Zener diode on the AC side can cause two different voltage drops for the corresponding circuit, depending on a phase of an oscillator voltage. By placing the Zener diode on the DC side, however, voltage regulation is achieved with the reverse voltage of the Zener diode.

Broadly speaking, the power receiving unit may be capable of operating with a greater voltage than other components of an implant, such as a power management integrated circuit (PMIC) of the implant. By way of example, the power receiving unit may be exposed to and capable of operating within ranges of a voltage up to 20 volts while the PMIC may be capable of accepting 6.5 volts before latching. To prevent the PMIC from latching, a Zener diode may be used as a form of clamping to ensure that the voltage applied to the PMIC does not exceed 6.5 volts. When a Zener diode is used alone though, the Zener diode may be at risk of overvoltage, e.g., clamping excessive voltage to protect the PMIC may stress the Zener diode causing it to break. This is because the power receiving unit may only measure 6.5 volts—even when the Zener diode in the example is exposed to more than 6.5 volts—since the example Zener diode clamps at 6.5 volts. Further, as the induced voltage on the power receiving unit increases, the current through the Zener diode also increases—even though the voltage across the Zener diode remains constant. To prevent the Zener diode from breaking, aspects involve triggering an OVP condition in response to detecting that the Zener diode is under excessive stress.

To detect this excessive stress, a thermistor may be incorporated into the configuration so that it is disposed next to the Zener diode, e.g., so that the Zener diode and the thermistor are within temperature-measuring proximity. As used herein, the term “temperature-measuring proximity” refers to a spatial relationship between a thermistor and Zener diode. This spatial relationship may be defined by a distance within which the thermistor is capable of accurately measuring a temperature of a Zener diode. In this configuration, a temperature of the Zener diode may be utilized to determine when the power receiving unit is in an OVP condition. Broadly speaking, the temperature measured by the thermistor correlates to the voltage stress on the Zener diode. This correlation may be observed, in part, because the environment where the implant is located (e.g., in the body) generally has a consistent temperature due to blood heatsinking. In other ambient environments without a generally consistent temperature, the measured temperature and the voltage stress may not correlate in such a reliable manner.

When the measured temperature indicates that the Zener is overstressed, the system may respond in a variety of different ways to reduce the stress on the Zener diode and prevent an overvoltage condition. By way of example, the power receiving unit may respond locally, such as by triggering another OVP circuit (e.g., as one example another OVP circuit may short a resonator terminal of the power receiving unit to ground). The power receiving unit may also notify a wireless power transmitter, providing the wireless power for the implant, of the condition thereby causing the wireless power transmitter to regulate (e.g., reduce or cease) power transmission.

These and other aspects of Zener OVP with a thermal trigger are described below in the context of an example environment, example arrangements of a Zener diode and thermistor, and techniques. Any reference made with respect to the example environment or Zener diode and thermistor arrangements, or elements thereof, is by way of example only and is not intended to limit any of the aspects described herein.

### Example Environment

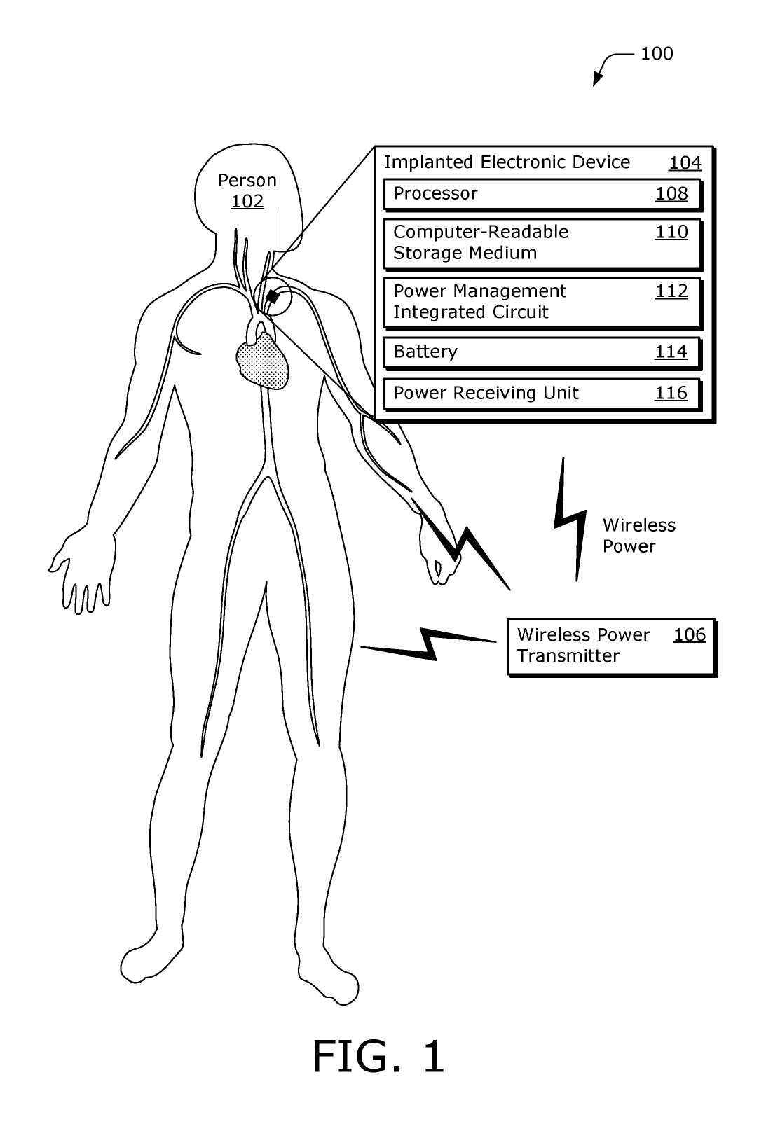

FIG. 1 illustrates an example environment 100, which includes a person 102 in which an implanted electronic device 104 has been surgically implanted. The example environment also includes a wireless power transmitter 106, which is configured to supply power wirelessly to the implanted electronic device 104. The implanted electronic device 104 may be implemented as any suitable computing or electronic device that is implanted in the person 102 and capable of being powered with the wireless power transmitted by the wireless power transmitter 106. Examples of the implanted electronic device 104 include implants to release metered doses of medication, implants to stimulate bodily tissue (e.g., nerves), implants for managing reproduction, implants to monitor specific biochemical conditions, and so on. Electronic devices other than medical-based implants may also be contemplated within the techniques described herein, such as personal communication devices, identification devices, location tracking devices, and so forth. Accordingly, the implanted electronic device 104 may correspond to a variety of different implanted computing or electronic devices without departing from the spirit or scope of the techniques described herein.

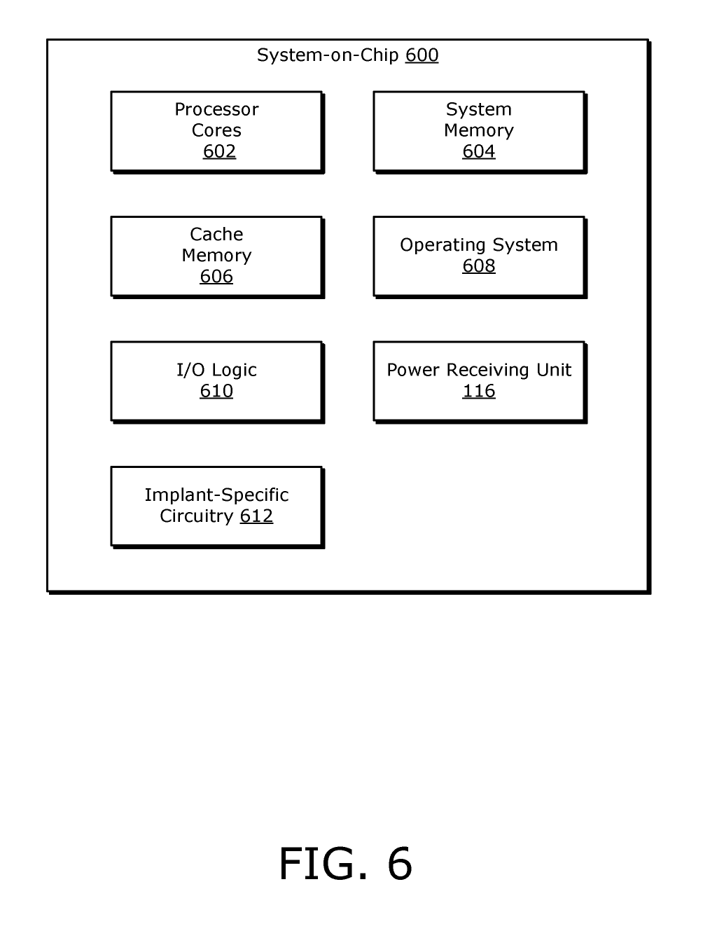

The implanted electronic device 104 includes a processor 108. In the example, the implanted electronic device 104 also includes computer-readable storage medium 110 (CRM 110). The processor 108 may include any type of processor, such as an application processor or multi-core processor, configured to execute processor-executable code stored by the CRM 110. The CRM 110 may include any suitable type of data storage media, such as volatile memory (e.g., random access memory (RAM)), non-volatile memory (e.g., Flash memory), optical media, magnetic media (e.g., disk or tape), and the like. In the context of this disclosure, the CRM 110 is implemented to store instructions, data, and other information of the implanted electronic device 104, and thus does not include transitory propagating signals or carrier waves. Further, although the implanted electronic device 104 is illustrated with the CRM 110, in some aspects the implanted electronic device 104 may instead or additionally be implemented using a system-on-chip (SoC) as further described in relation to FIG. 6.

Although not depicted in the example, the implanted electronic device 104 may also include data interfaces to provide connectivity to respective networks and other electronic devices connected therewith. Such data interfaces may comprise wired data interfaces (that are usable to connect with the implanted electronic device 104 before it is implanted into a body, during a surgical procedure in which the implanted electronic device 104 is exposed, when the implanted electronic device 104 has been removed from the body, and so on), wireless data interfaces, or any suitable combination thereof. Alternately or additionally, the wireless interfaces may include a modem or radio configured to communicate over a wireless network, such as a wireless LAN, peer-to-peer (P2P), cellular network, and/or wireless personal-area-network (WPAN).

The implanted electronic device 104 also includes power management integrated circuit 112 (PMIC 112), battery 114, and power receiving unit 116. The PMIC 112 is capable of managing power for the implanted electronic device 104, including managing electrical power conversion and power control functions. In conjunction with the power receiving unit 116, the PMIC 112 may manage the power received by the implanted electronic device 104 via the power receiving unit 116. By way of example, the PMIC 112 may be capable of conditioning the power received via the power receiving unit 116 so that the power is usable by the implanted electronic device 104 to carry out its corresponding functionality

The battery 114 represents functionality to store power received via the power receiving unit 116 for later use. In some aspects, the power received by the power receiving unit 116 may be fed to the battery 114, and the implanted electronic device 104 may draw power for operation from the battery 114. In other aspects, the implanted electronic device 104 may draw power for operation directly from the power receiving unit 116 and rely on the battery 114 solely when the power received directly from the power receiving unit 116 is not enough to function properly. In both cases, the implanted electronic device 104 may be configured to use power stored in the battery 114 for operation.

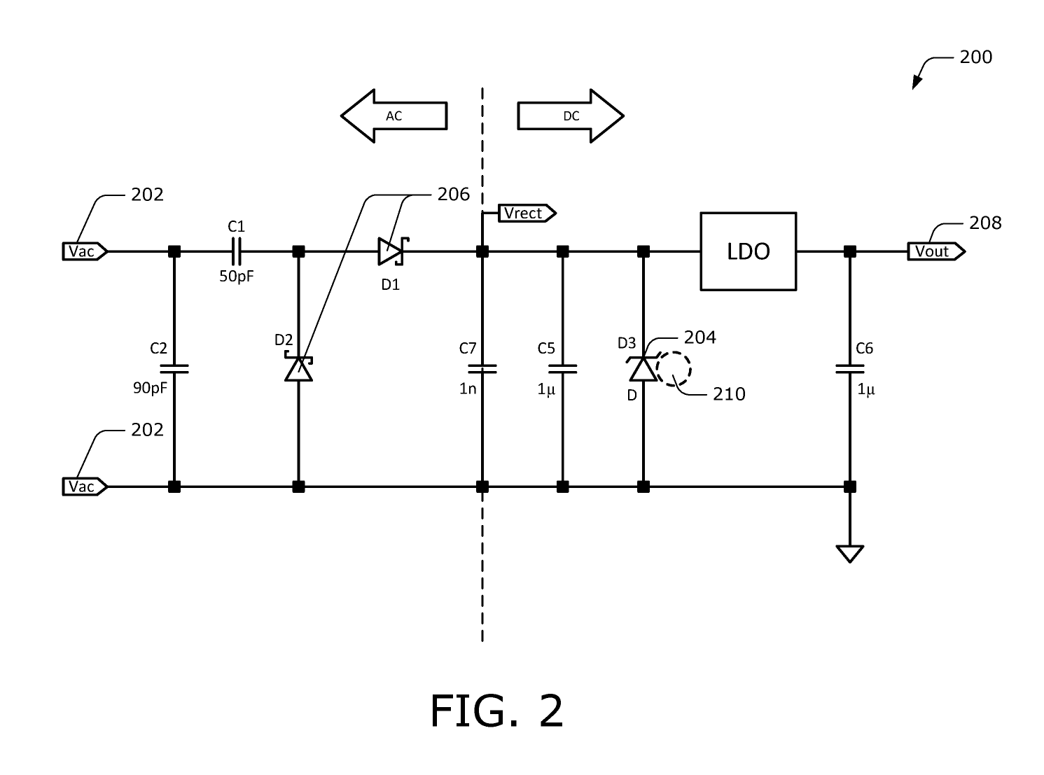

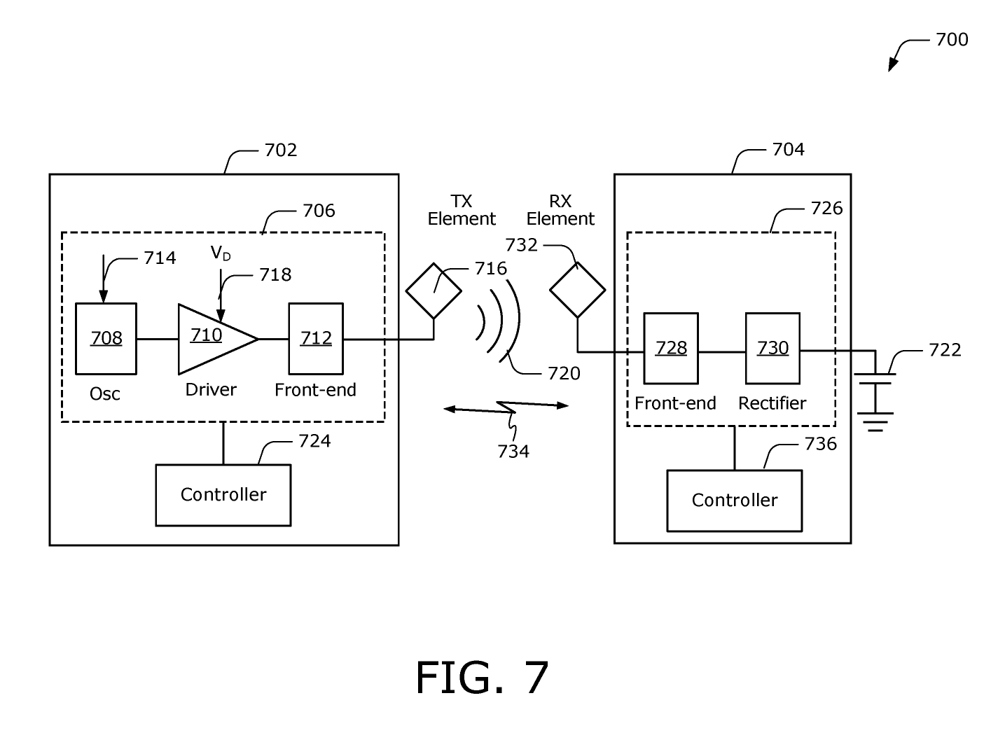

Broadly speaking, the power receiving unit 116 represents functionality of the implanted electronic device 104 to receive power wirelessly from the wireless power transmitter 106. The power receiving unit 116 may be configured in accordance with the wireless power transfer system discussed in relation to FIG. 7. In one or more aspects, the power receiving unit 116 may include a resonator configured to resonate when exposed to electromagnetic fields or magnetic fields having a certain frequency or certain frequencies, including electromagnetic or magnetic fields produced by the wireless power transmitter 106. Accordingly, the wireless power transmitter 106 may be configured to generate electromagnetic or magnetic fields having a certain frequency and a resonator of the power receiving unit 116 configured to resonate when exposed to electromagnetic or magnetic fields with that frequency. Such resonating causes current to flow through the power receiving unit 116. In accordance with the described aspects, the power receiving unit 116 may be configured with a rectification circuit, such as the one depicted in FIG. 2. The rectification circuit is capable of converting the power generated by the resonator into a form that is usable by the implanted electronic device 104. This may involve rectifying the power from alternating current (AC) to direct current (DC), lowering a voltage, and so on.

The rectification circuit ensures that the components of the implanted electronic device 104 used to carry out its functionality receive power in a form for which they are designed. This prevents those components from becoming stressed beyond their designed operating capabilities and breaking—causing the implant to fail, or worse injuring the person 102. To do so, the rectification circuit may cutoff power above a certain voltage. The term “clamping” refers to cutting off the voltage above a predefined level. By clamping the power above a certain voltage, the rectification circuit provides “overvoltage protection” (OVP) for the components of the electronic device. In accordance with the described aspects, the rectification circuit includes a Zener diode to clamp a voltage of the power generated by the power receiving unit 116's resonator. As discussed above and below, the Zener diode is configured to clamp the voltage at certain level, such as 6.5 volts as discussed in the example above. The Zener diode thus provides overvoltage protection for components of the implanted electronic device 104.

Although the power output from the Zener diode is clamped at the predefined voltage, the power input to the Zener diode can exceed the predefined voltage. By way of example, the power received by the resonator of the power receiving unit 116 and fed into the Zener diode of rectification circuit can surpass the predefined voltage. Further, use of the Zener diode may prevent the power receiving unit 116 from detecting how far the voltage is above the predefined voltage. Instead, the power receiving unit 116 may measure the voltage at the predefined level because the Zener diode is clamping. Excessive application of power with a voltage above the predefined voltage can stress the Zener diode and cause it to break. The techniques described herein are used to detect that the Zener diode is under excessive stress and to trigger a corresponding OVP condition.

In accordance with one or more aspects, the described techniques utilize a thermal trigger with the Zener diode to provide OVP. For instance, a thermistor can be placed next to the Zener diode to measure its temperature. The measured temperature is usable to determine when the power receiving unit 116 is in an OVP condition. In an implant, the temperature measured by the thermistor correlates to the voltage stress on the Zener diode. In other words, the temperature measured by the thermistor can be used to determine that an amount of voltage applied to the Zener diode is greater than the predefined voltage. This is because the environment in which the implanted electronic device 104 is disposed generally has a steady temperature, e.g., due to blood heatsinking, flesh, or muscle. In scenarios where the ambient environment does not have a steady temperature, such as some scenarios outside a body, the temperature measured by a thermistor and voltage stress on a Zener diode within temperature-measuring proximity may not correlate in a reliable manner.

The power receiving unit 116 also represents functionality to respond to detection of an overstressed Zener diode. By way of example, the power receiving unit 116 may carry out a remedial course of action when the temperature measured by the thermistor surpasses a threshold temperature. The threshold temperature can be set at a level that prevents the Zener diode from being stressed to the point of failure and/or breaking. Continuing with the discussion of the power receiving unit 116's remedial course of action, the power receiving unit 116 may in some scenarios act locally and trigger an OVP circuit, which may short resonator terminals of the power receiving unit 116 to ground. In addition or alternately, the power receiving unit 116 may trigger a remote course of action by notifying the wireless power transmitter 106 about the OVP condition. The wireless power transmitter 106 may then initiate one or more remedial actions of its own, including lowering a voltage of the power transmitted, ceasing transmission of the power, triggering a system reset, and so forth. How a power receiving unit 116 may be specifically implemented to provide Zener OVP with a thermal trigger is described in greater detail below.

### Example Power Receiving Units

FIG. 2 illustrates an example rectification circuit of the power receiving unit 116 from FIG. 1 in accordance with one or more aspects at 200. In particular, the rectification circuit includes resonator terminals 202, Zener diode 204, rectifier 206, and output terminal 208. The example rectification circuit also includes a variety of other components for conditioning power received at the resonator terminals 202 from the power receiving unit 116's resonator (not shown). By way of example, the example rectification circuit is also depicted with a 90-picofarad capacitor, a 50-picofarad capacitor, two Schottky diodes, a nanofarad capacitor, two microfarad capacitors, a low-dropout (LDO) regulator, and ground terminal. The rectification circuit may include more, fewer, or simply different power-conditioning components than those depicted without departing from the spirit or scope of the described aspects, including a field effect transistor (FET) configured to short the resonator terminals 202 to ground.

Broadly speaking, the resonator terminals 202 are capable of receiving power from a resonator of the power receiving unit 116 (FIG. 1). In general, the resonator is configured to resonate when exposed to a wireless field generated by the wireless power transmitter 106—the wireless field causing current to flow through the power receiving unit 116 thereby powering the implanted electronic device 104. In particular, in an implementation that relies on resonance, the resonator resonates when exposed to a wireless field having a certain frequency or that are within a range of frequencies for which the resonator is designed. Further, the wireless field causes flow of an alternating current (AC) at the resonator terminals 202. The resonator is thus configured to provide the AC power. This is illustrated at the resonator terminals 202 using the label “Vac” which indicates an AC voltage.

As discussed above and below, the rectification circuit also includes the Zener diode 204 to clamp a voltage of the power flowing through the rectification circuit at a predefined voltage. As mentioned above, the power receiving unit 116 may be exposed to a range of voltages that is greater than the other components of the implanted electronic device 104, such as the components of the implanted electronic device 104 that carry out its corresponding functionality. By way of example, the power receiving unit 116 may be capable of operating with a voltage of up to 20 volts while the components for carrying out the implanted electronic device 104's functionality may be capable of accepting 6.5 volts before latching. To prevent this latching, the Zener diode 204 is used to clamp the voltage to ensure that the voltage applied to those components does not exceed the predefined voltage level, e.g., 6.5 volts in the just-discussed example.

The techniques herein incorporate the Zener diode 204 on a direct current (DC) side of the rectification circuit rather than on an AC side. The rectifier 206 represents functionality to rectify the AC power received at the resonator terminals 202 to DC power, which is usable by the implanted electronic device 104 to carry out its corresponding functionality. Thus, the current flowing through the rectification circuit between the resonator terminals 202 and the rectifier 206 is AC, while the current flowing through the rectification circuit beyond the rectifier 206 is DC. Accordingly, the AC side of the rectification circuit corresponds to those portions through which the AC power flows and the DC side corresponds to those portions through which the DC power flows. The AC side and DC side of the example rectification circuit are indicated, respectively, with the arrows in FIG. 2 labeled “AC” and “DC.” By inserting the Zener diode 204 on the DC side, rather than on the AC side, the rectification circuit is capable of achieving voltage regulation with solely the reverse voltage of the Zener diode 204. This contrasts with utilizing Zener diodes on an AC side, which can cause two different voltage drops for the corresponding circuit depending on a phase of an oscillator voltage.

The output terminal 208 is capable of outputting power that is conditioned for use by the implanted electronic device 104. In particular, the power output at the output terminal 208 meets operating specifications for other functional components of the implanted electronic device 104. To the extent that the example rectification circuit includes the Zener diode 204 and the rectifier 206, the power output by the output terminal 208 is at least rectified from AC to DC and clamped at the predefined level for which the Zener diode 204 is designed.

Although the Zener diode 204 clamps the power at the predefined voltage, the Zener diode 204 may nevertheless receive power having a greater voltage. Overexposure to power having a voltage in excess of the predefined voltage can stress the Zener diode 204 and cause it to break. This corresponds to an overvoltage condition for the Zener diode 204. To prevent stressing the Zener diode 204 to the point of failure, a thermal trigger is used in accordance with one or more aspects. In particular, the Zener diode 204 may be within temperature-measuring proximity of a thermistor. Position 210 represents a physical location of the power receiving unit 116 where a thermistor may be inserted to be temperature-measuring proximal with the Zener diode 204. By being temperature-measuring proximal to the Zener diode 204 at the position 210, the thermistor may accurately measure a temperature of the Zener diode 204. As noted above, the temperature measured by the thermistor correlates to the voltage on the Zener diode—the correlation is reliable because the implant is in a body with a generally consistent temperature. If the Zener diode 204 and the thermistor are built into an application-specific integrated circuit (ASIC), the correlation may be even higher than implementations in which the Zener diode and thermistor are not part of an ASIC.

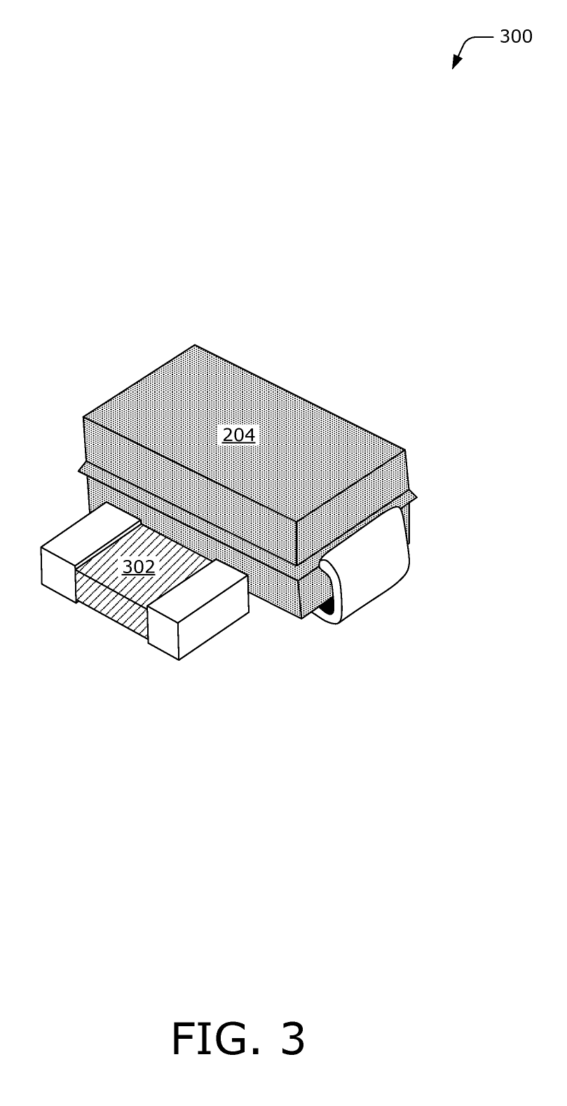

FIG. 3 illustrates an example of a Zener diode and a thermistor in temperature-measuring proximity to provide Zener OVP with a thermal trigger in accordance with one or more aspects at 300. In particular, the illustrated example depicts a three dimensional (3D) view of the Zener diode 204 and thermistor 302.

Although temperature-measuring proximal to the Zener diode 204, the thermistor 302 may not be included as part of the rectification circuit. Instead, the thermistor 302 may be included as part of a thermal triggering circuit (not shown) of the power receiving unit 116. By way of example, the thermistor 302 may be incorporated in the power receiving unit 116 at the position 210, but not be electrically coupled to the example rectification circuit. In accordance with one or more aspects, the thermal triggering circuit may include a microcontroller configured to act on the temperature measured by the thermistor 302, including carrying out remedial actions to provide OVP when the measured temperature exceeds a threshold temperature, notifying other components of the power receiving unit 116 when the threshold is exceeded so those other components can carry out remedial actions, and so forth. It should be appreciated that the Zener diode 204 and the thermistor 302 may not be drawn to scale. Rather, the example illustrated generally at 300 may simply convey an approximate disposition of the Zener diode 204 and the thermistor 302 in temperature-measuring proximity. The relative sizes of the Zener diode 204 and the thermistor 302 also may not be accurately scaled in the drawing. Indeed, the relative size and position of the Zener diode 204 and the thermistor 302 may vary in actual implementations without departing from the spirit or scope of the techniques described herein.

Returning to the discussion of remedial actions taken when an OVP condition is detected, in accordance with one or more aspects. The power receiving unit 116 is further configured to initiate the remedial actions when the temperature measured by the thermistor 302 indicates an OVP condition for the Zener diode 204.

As discussed above, the power receiving unit 116 may take local remedial actions or may communicate with the wireless power transmitter 106, causing the wireless power transmitter 106 to take actions to remedy the OVP condition. Broadly speaking, remedial OVP actions can include activating a circuit that reduces a voltage applied to the Zener diode 204. By way of example, the rectification circuit may include a field effect transistor (FET) configured to short the resonator terminals 202 to ground, as discussed in relation to FIG. 2. Accordingly, the power receiving unit 116 may act locally by triggering the FET to short the resonator terminals 202 to ground. The power receiving unit 116 may keep the FET triggered so that the resonator terminals 202 short to ground until a temperature measured by the thermistor 302 decreases back below a threshold temperature—at which point the power receiving unit may open the FET to discontinue shorting the resonator terminals to ground. This threshold temperature may be a temperature at which the Zener diode may safely operate without breaking, and it may be the same or different than the threshold temperature indicative of the OVP condition. By way of example, this threshold temperature indicative of safe operation may be lower than the threshold indicative of the OVP condition. This can ensure that the Zener diode 204 operates safely for at least some period of time before experiencing another OVP condition. Although an FET is specifically discussed, the power receiving unit 116 may trigger other OVP circuits to remedy detected OVP conditions in the spirit and scope of the described techniques. The power receiving unit 116 may also remedy detected OVP conditions by initiating yet other local actions.

Alternately or in addition, the power receiving unit 116 may communicate with the wireless power transmitter 106 to take actions to remedy the OVP condition. By way of example, the power receiving unit 116 may notify the wireless power transmitter 106 of a detected OVP condition. Based on the notification, the wireless power transmitter 106 may initiate a reset so that when reset, the wireless power transmitter 106 transmits wireless power at an appropriate level. The wireless power transmitter 106 may also adjust an amount of power being transmitted to the appropriate level. If a temperature of the Zener diode 204 is continuously monitored, for instance, the power receiving unit 116 can notify the wireless power transmitter 106 of the continuously monitored temperature. The wireless power transmitter 106 can adjust the amount of power transmitted based on the temperature, e.g., the wireless power transmitter 106 may determine to transmit no power, transmit a relatively low level of power while a temperature of the Zener diode 204 decreases, transmit a maximal amount of power when a temperature of the Zener diode 204 falls below the above-discussed threshold temperature indicating that the Zener diode 204 has returned to a safe operating temperature, and so forth.

The power receiving unit 116 may communicate with the wireless power transmitter 106 in a variety of different ways, including using in-band and out-of-band communication. In-band communication techniques may utilize the resonator of the power receiving unit 116 to communicate different signals to the wireless power transmitter 106, such as signals indicating that an OVP condition has been detected, indicating a measured temperature of the Zener diode 204, and so on. Out-of-band communication technique may utilize communication-dedicated hardware, such as a modem or radio with which the implanted electronic device 104 is configured to communicate over a wireless personal-area-network (WPAN). Out-of-band communication techniques may correspond to different frequencies (or a different frequency band) than the wireless power transmitted by the wireless power transmitter 106.

In some aspects, communications between the implanted electronic device 104 and the wireless power transmitter 106 regarding detected OVP conditions may be configured according to a communication standard for implants and wireless power transmitters. Such a standard may specify a format of communications, for instance, such as specifying that a communication indicating an OVP condition include a measured temperature for the Zener diode and a timestamp. Alternately or in addition, the standard may specify how implants are to detect and handle OVP conditions, how a wireless power transmitter is to respond when notified of a detected OVP condition, and so forth. By utilizing a standard, implants developed by different implant providers may predictably handle detected OVP conditions. Further, this may allow multiple implants developed by different providers to be surgically implanted into a body and powered by a single wireless power transmitter. The wireless power transmitter may be configured to handle OVP conditions detected in a single one of the multiple implanted devices according to the standard.

Techniques of Zener OVP with a Thermal Trigger

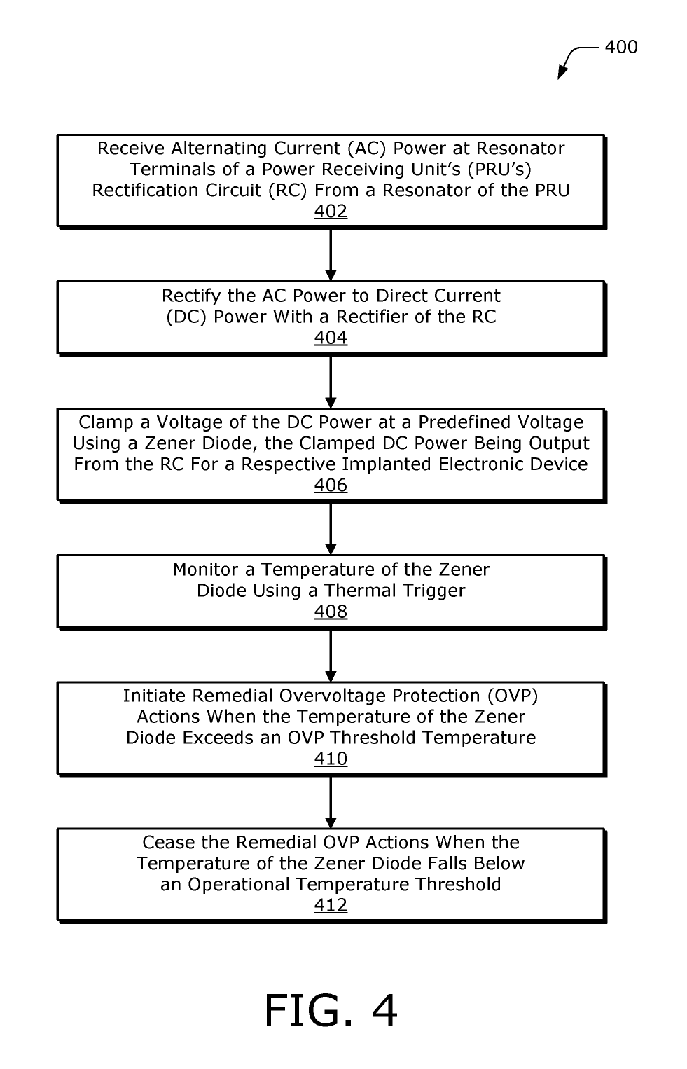

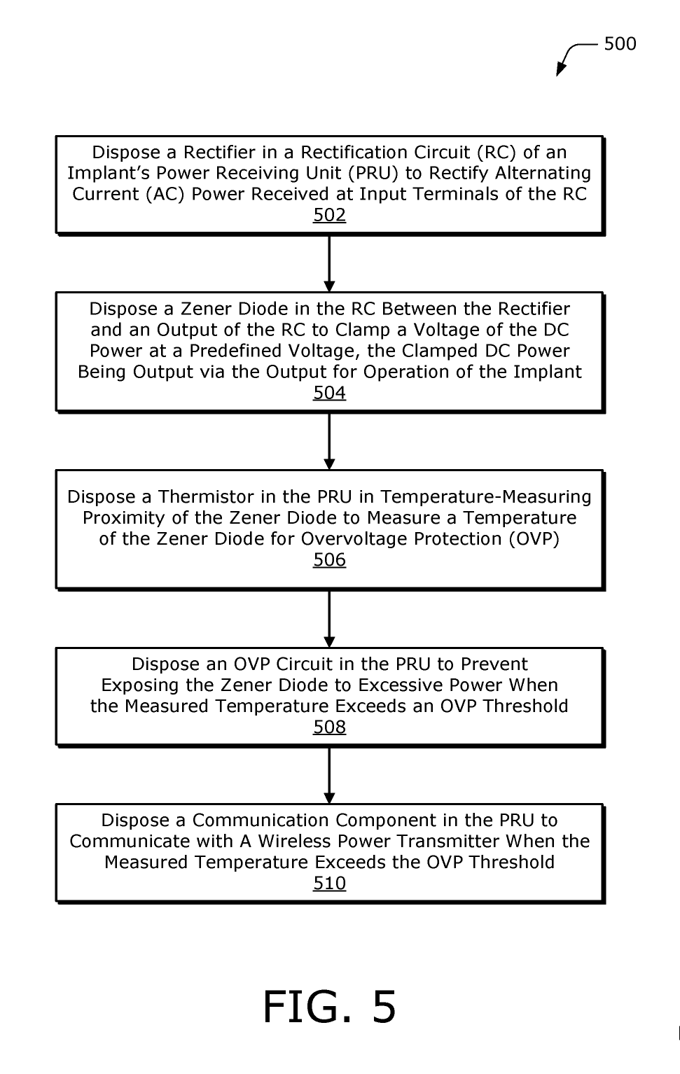

The following techniques of Zener OVP with a thermal trigger may be implemented using any of the previously described power receiving units of the example environment. The techniques may also involve powering an implant configured like the implanted electronic device 104 of the example environment or the system-on-chip described with reference to FIG. 6. Reference to entities, such as the power receiving unit 116 or the implanted electronic device 104, is made by example only and is not intended to limit the ways in which the techniques can be implemented. The techniques are described with reference to example methods illustrated in FIGS. 4 and 5. The example methods are depicted as respective sets of operations or acts that may be performed using the entities described herein and/or any suitable components which provide means for implementing one or more of the operations. The depicted sets of operations illustrate a few of the many ways in which the techniques may be implemented. As such, operations of a method may be repeated, combined, separated, omitted, performed in alternate orders, performed concurrently, or used in conjunction with another method or operations thereof

FIG. 4 illustrates an example method 400 of providing Zener OVP with a thermal trigger for an implanted electronic device in a body, including operations performed by the power receiving unit 116. In the following discussion, the power receiving unit 116 and other entities of the example environment 100 may provide means for implementing one or more of the operations described.

At 402, the method includes receiving alternating current (AC) power at resonator terminals of a power receiving unit's rectification circuit from a resonator of the power receiving unit. The power receiving unit may be included as part of an implanted electronic device. Additionally, the resonator terminals receive the AC power from a resonator of the power receiving unit, which is configured to resonate when exposed to electromagnetic or magnetic fields having a certain frequency or within a range of frequencies.

By way of example, consider FIG. 2, which illustrates an example rectification circuit in accordance with one or more aspects generally at 200. In this example, the rectification circuit is incorporated in the power receiving unit 116 of the implanted electronic device 104 depicted in FIG. 1. Further, the resonator terminals 202 receive AC power from a resonator of the power receiving unit 116. This resonator is configured to resonate when exposed to electromagnetic or magnetic fields transmitted by the wireless power transmitter 106, which it transmits with the certain frequency or range of frequencies to supply the implanted electronic device 104 with power.

At 404, the method includes rectifying the AC power to direct current (DC) power with a rectifier of the rectification circuit. By way of example, the rectifier 206 rectifies the AC power received by the resonator terminals 202 to DC power. Accordingly, power flowing through the rectification circuit between the resonator terminals 202 and the rectifier 206 is the AC power and the power flowing through the rectification circuit from the rectifier 206 to the output terminal 208 is the DC power. The portion of the rectification circuit through which the AC power flows thus corresponds to the “AC side” of the circuit and the portion of the rectification circuit through which the DC power flows thus corresponds to the “DC side” of the circuit.

At 406, the method includes clamping a voltage of the DC power at a predefined voltage using a Zener diode of the rectification circuit. The clamped DC power is output from the rectification circuit for use by the implanted electronic device. By way of example, the Zener diode 204 clamps a voltage of the DC power at a predefined voltage. The predefined voltage may correspond to a voltage level at which downstream electrical components (such as the PMIC 112) of the implanted electronic device 104 are configured to operate. By clamping the voltage at the predefined level, the Zener diode 204 prevents these downstream components from experiencing an overvoltage condition—which can break the components and, in some cases, even injure the person 102 in which the implanted electronic device 104 is surgically implanted.

Further, by clamping the voltage on the DC side of the rectification circuit, rather than on the AC side, the rectification circuit achieves voltage regulation solely with a reverse voltage of the Zener diode 204. Techniques that utilize Zener diodes on an AC side of a circuit, however, can cause two different voltage drops for the corresponding circuit depending on a phase of an oscillator voltage.

Continuing with the example in which the Zener diode clamps the DC power at the predefined voltage, doing so excessively can stress the Zener diode 204 causing it to break. Moreover, due to the Zener diode 204's clamping, the power receiving unit 116 may merely measure a voltage at the predefined level, even though the Zener diode 204 clamps power having significantly higher voltages. Using solely a voltage measurement, therefore, the power receiving unit 116 may not be able to detect when the Zener diode 204 is overstressed. The temperature of the Zener diode 204, however, correlates to the voltage stress on the Zener diode 204, and thus can be used to detect when the Zener diode 204 reaches an OVP condition. This correlation between temperature and stress on the Zener diode 204 can be observed because the environment in which the implanted electronic device 104 is disposed generally has a steady temperature, e.g., due to blood heatsinking, flesh, or muscle. When the thermistor 302 and the Zener diode 204 are built into an ASIC, there may be a more positive correlation between the measured temperature and the voltage stress on the Zener diode 204 than in other implementations.

At 408, the method includes monitoring the temperature of the Zener diode using a thermal trigger. By way of example, consider FIG. 3, which illustrates the Zener diode 204 of the rectification circuit and the thermistor 302. In this example, the thermistor 302 is temperature-measuring proximal to the Zener diode 204, e.g., by inserting it into the power receiving unit 116 at the position 210. Due to the temperature-measuring proximity of the thermistor 302 and the Zener diode 204, the temperature measured by the thermistor 302 corresponds to the temperature of the Zener diode 204.

At 410, the method includes initiating remedial overvoltage protection (OVP) actions when the temperature of the Zener diode exceeds an OVP threshold temperature. By way of example, when the Zener diode 204 clamps DC power with voltage that exceeds the predefined voltage for an excessive period of time, the Zener diode 204's temperature rises. As the Zener diode 204 continues to clamp the voltage, the temperature rises above an OVP threshold, for instance. The thermistor 302 measures the temperature of the Zener diode 204 that exceeds the OVP threshold. Responsive to this, the power receiving unit 116 initiates one or more remedial OVP actions. As discussed above in more detail, the power receiving unit 116 initiates local remedial OVP actions and/or communicates with the wireless power transmitter 106 so the wireless power transmitter 106 can initiate remedial OVP actions.

In connection with an exemplary remedial action, a circuit is activated that reduces a voltage applied to the Zener diode 204. In connection with another local remedial action, the power receiving unit 116 triggers an OVP circuit such as a field effect transistor (FET) to short the resonator terminals 202 to ground. An example of communicating with the wireless power transmitter 106 includes notifying the wireless power transmitter 106 of the detected OVP condition. Based on the notification, the wireless power transmitter 106 resets to transmit an adjusted level of wireless power.

At 412, the method includes ceasing the remedial OVP actions when the temperature of the Zener diode falls below an operational temperature threshold. The operational temperature threshold is a temperature below which the Zener diode 204 can operate without risk of breaking. By way of example, the thermistor 302 measures that the temperature of the Zener diode 204 falls below the operational temperature threshold. Responsive detecting that the Zener diode 204's temperature falls below the operational temperature threshold, the power receiving unit 116 ceases the remedial OVP actions initiated at 410. For instance, the power receiving unit 116 discontinues shorting the resonator terminals 202 to ground. In accordance with the discussed aspects, the power receiving unit 116 can also communicate with the wireless power transmitter 106 to indicate that the temperature of the Zener diode 204 indicates it can be safely used again for clamping. The wireless power transmitter 106 can thus adjust the wireless power transmitted accordingly.

FIG. 5 illustrates an example method 500 of configuring an implanted electronic device to provide Zener OVP with a thermal trigger. In the following discussion, the power receiving unit 116 and other entities of example environment 100 may provide means for implementing one or more of the operations described.

At 502, the method includes disposing a rectifier in a rectification circuit of an implant's power receiving unit to rectify alternating current (AC) power received from input terminals of the rectification circuit to direct current (DC) power. By way of example, consider again FIG. 2, which illustrates an example rectification circuit in accordance with one or more aspects generally at 200. In aspects, the rectification circuit of FIG. 2 is included as part of the power receiving unit 116 of the implanted electronic device 104. In this example, the rectifier 206 is disposed in the example rectification circuit to rectify AC power received from the resonator terminals 202 to DC power.

At 504, the method includes disposing a Zener diode in the rectification circuit between the rectifier and an output of the rectification circuit to clamp a voltage of the DC power at a predefined voltage. The clamped DC power being output via the output of the rectification circuit for operation of the implant. By way of example, the Zener diode 204 is disposed in the rectification circuit between the rectifier 206 and the output terminal 208. Here, the Zener diode 204 clamps a voltage of the DC power that is output by the rectifier 206 at a predefined voltage. The output terminal 208 outputs the clamped DC power for use by the implanted electronic device 104 to carry out its corresponding functionality. The components of the implanted electronic device 104 that are used to carry out the corresponding functionality are designed to utilize power meeting specified operational criteria, e.g., the power has direct rather than alternating current, has a certain voltage (at or below the predefined voltage), and so forth.

At 506, the method includes disposing a thermistor in the power receiving unit so that the Zener diode is within temperature-measuring proximity of the thermistor to measure a temperature of the Zener diode for overvoltage protection (OVP). By way of example, the thermistor 302 is temperature-measuring proximal in the power receiving unit 116 to the Zener diode 204, e.g., by inserting the thermistor 302 in the power receiving unit 116 at the position 210. The thermistor 302 measures the temperature of the Zener diode 204 to for OVP—to detect when a temperature of the Zener diode 204 indicates excessive stress on the Zener diode 204.

At 508, the method optionally includes disposing an OVP circuit in the power receiving unit to prevent exposing the Zener diode to excessive power when the measured temperature exceeds an OVP threshold. By way of example, a field effect transistor (FET) is included in the power receiving unit 116 to prevent exposing the Zener diode 204 to excessive power when the measured temperature exceeds the OVP threshold. The FET can be triggered by the power receiving unit 116 when the measured temperature exceeds the OVP threshold. Triggering the FET shorts the resonator terminals 202 to ground effective to prevent power from flowing through the rectification circuit to the Zener diode 204 and further stress on the Zener diode 204.

At 510, the method optionally includes disposing a communication component in the power receiving unit to communicate with a wireless power transmitter when the measured temperature exceeds the OVP threshold. In some aspects, the power receiving unit 116 may not initiate local remedial actions, such as described at 508. Instead or in addition to local remedial actions, the power receiving unit 116 may communicate with the wireless power transmitter 106 to remedy OVP conditions. In accordance with one or more aspects, a communication component is disposed in the power receiving unit 116 to communicate with the wireless power transmitter 106 when the measured temperature exceeds the OVP threshold. As discussed in more detail above, communication components may be configured for in-band communication or out-of-band communication with the wireless power transmitter 106.

The described aspects provide power to the implanted electronic device 104 in a form for which its components are designed. This allows the implanted electronic device 104 to carry out the functionality for which it was designed using the wireless power received from the wireless power transmitter 106.

System-On-Chip

FIG. 6 illustrates an example system-on-chip 600, which includes components capable of implementing aspects of Zener OVP with a thermal trigger. The system-on-chip 600 may be implemented as, or in, any suitable electronic device in a body, such as implants to release metered doses of medication, implants to stimulate bodily tissue (e.g., nerves), implants to monitor specific biochemical conditions, and so on, or any other device that may utilize power transmitted wirelessly from a transmitter outside the body.

The system-on-chip 600 may be integrated with, a microprocessor, storage media, I/O logic, data interfaces, logic gates, a transmitter, a receiver, circuitry, firmware, software, or combinations thereof to provide communicative or processing functionalities. The system-on-chip 600 may include a data bus (e.g., cross bar or interconnect fabric) enabling communication between the various components of the system-on-chip. In some aspects, components of the system-on-chip 600 may interact via the data bus to implement aspects of Zener OVP with a thermal trigger.

In this particular example, the system-on-chip 600 includes processor cores 602, system memory 604, and cache memory 606. The system memory 604 or the cache memory 606 may include any suitable type of memory, such as volatile memory (e.g., DRAM), non-volatile memory (e.g., Flash), and the like. The system memory 604 and the cache memory 606 are implemented as a storage medium, and thus do not include transitory propagating signals or carrier waves. The system memory 604 can store data and processor-executable instructions of the system-on-chip 600, such as operating system 608 and other applications. The processor cores 602 execute the operating system 608 and other applications from the system memory 604 to implement functions of the system-on-chip 600, the data of which may be stored to the cache memory 606 for future access. The system-on-chip 600 may also include I/O logic 610, which can be configured to provide a variety of I/O ports or data interfaces for inter-chip or off-chip communication.

The system-on-chip 600 also includes the power receiving unit 116 and implant-specific circuitry 612, which may be embodied separately or combined with other components described herein. For example, the power receiving unit 116 may be integral with the PMIC 112 or the battery 114 as described with reference to FIG. 1. The implant-specific circuitry 612 can be implemented to carry out the functionality specific to an implant.

The implant-specific circuitry 612 may also be integrated with other components of the system-on-chip 600, such as the cache memory 606, a memory controller of the system-on-chip 600, or any other signal processing, modulating/demodulating, or condition sections within the system-on-chip 600. The implant-specific circuitry 612 and other components of the system-on-chip 600 may be implemented as hardware, fixed-logic circuitry, firmware, or a combination thereof that is implemented in association with the I/O logic 610 or other signal processing circuitry of the system-on-chip 600.

### Wireless Power Transfer System

FIG. 7 illustrates an example wireless power transfer system 700, which includes components capable of implementing aspects of Zener OVP with a thermal trigger. The system 700 includes a transmitter 702 and a receiver 704. The transmitter 702 may include transmit circuitry 706 having an oscillator 708, a driver circuit 710, and a front-end circuit 712. The oscillator 708 may be configured to generate an oscillator signal at a desired frequency that may adjust in response to a frequency control signal 714. The oscillator 708 may provide the oscillator signal to the driver circuit 710. The driver circuit 710 may be configured to drive the power transmitting element 716 at, for example, a resonant frequency of the power transmitting element 716 based on an input voltage signal (VD) 718. The driver circuit 710 may be a switching amplifier configured to receive a square wave from the oscillator 708 and output a sine wave.

The front-end circuit 712 may include a filter circuit configured to filter out harmonics or other unwanted frequencies. The front-end circuit 712 may include a matching circuit configured to match the impedance of the transmitter 702 to the impedance of the power transmitting element 716. The front-end circuit 712 may include also a tuning circuit to create a resonant circuit with the power transmitting element 716. As a result of driving the power transmitting element 716, the power transmitting element 716 may generate a wireless field 720 to wirelessly output power at a level sufficient for charging a battery 722, or otherwise powering a load.

The transmitter 702 may further include a controller 724 operably coupled to the transmit circuitry 706 and configured to control one or more aspects of the transmit circuitry 706, or accomplish other operations relevant to managing the wireless transfer and Zener OVP with a thermal trigger. The controller 724 may be a micro-controller or a processor. The controller 724 may be implemented as an application-specific integrated circuit (ASIC). The controller 724 may be operably connected, directly or indirectly, to each component of the transmit circuitry 706. The controller 724 may be further configured to receive information from each of the components of the transmit circuitry 706 and perform calculations based on the received information. The controller 724 may be configured to generate control signals (e.g., the control signal 714) for each of the components that may adjust the operation of that component. As such, the controller 724 may be configured to adjust or manage the power transfer for Zener OVP with a thermal trigger based on a result of the operations it performs. The transmitter 702 may further include a memory (not shown) configured to store data, for example, such as instructions for causing the controller 724 to perform particular functions, such as those related to management of wireless power transfer.

The receiver 704 may include receive circuitry 726 having a front-end circuit 728 and a rectifier circuit 730. The front-end circuit 728 may include matching circuitry configured to match the impedance of the receive circuitry 726 to the impedance of the power receiving element 732. The front-end circuit 728 may further include a tuning circuit to create a resonant circuit with the power receiving element 732. The rectifier circuit 730 may generate a DC power output from an AC power input to charge the battery 722, as shown in FIG. 7, or provide power to some other load. The receiver 704 and the transmitter 702 may additionally communicate on a separate communication channel 734, e.g., Bluetooth™, ZigBee™, and cellular. The receiver 704 and the transmitter 702 may alternatively communicate via in-band signaling using characteristics of the wireless field 720.

Further, the receiver 704 may be configured to determine whether an amount of power transmitted by the transmitter 702 and received by the receiver 704 is appropriate for charging the battery 722 or powering a load. In certain embodiments, the transmitter 702 may be configured to generate a predominantly non-radiative field with a direct field coupling coefficient (k) for providing energy transfer. The receiver 704 may directly couple to the wireless field 720 and may generate an output power for storing or consumption by the battery 722 (or load), coupled to the output of the receive circuitry 726.

The receiver 704 may further include a controller 736 configured similarly to the transmit controller 724 as described above for one or more wireless power management aspects of the receiver 704. The receiver 704 may further include a memory (not shown) configured to store data, such as instructions for causing the controller 736 to perform particular functions, such as those related to management of wireless power transfer and Zener OVP with a thermal trigger. The transmitter 702 and receiver 704 may be separated by a distance and configured according to a mutual resonant relationship to minimize transmission losses between the transmitter 702 and the receiver 704.

The power transmitting element 716 and the power receiving element 732 may correspond to or be included as part of, respectively, the wireless power transmitter 106 and the power receiving unit 116 that utilize the Zener OVP with a thermal trigger described herein.

Although subject matter has been described in language specific to structural features or methodological operations, it is to be understood that the subject matter defined in the appended claims is not necessarily limited to the specific features or operations described above, including not necessarily being limited to the organizations in which features are arranged or the orders in which operations are performed.

What is claimed is:

1. An apparatus for providing overvoltage protection in connection with receiving power wirelessly from a wireless power transmitter, the apparatus comprising:
a rectifier disposed in a rectification circuit and configured to rectify alternating current (AC) power received at the rectification circuit to direct current (DC) power;
a Zener diode disposed in the rectification circuit between the rectifier and an output terminal of the rectification circuit, the Zener diode configured to clamp the DC power at a predefined voltage, the clamped DC power output at the output terminal being usable by an electronic device; and
a thermistor configured to measure a temperature of the Zener diode, the measured temperature correlating to voltage at the Zener diode due to clamping and triggering one or more remedial overvoltage protection actions when the measured temperature exceeds a threshold temperature.

2. The apparatus as recited in claim 1, wherein the electronic device is an implant intended for use in a body, the apparatus provides the overvoltage protection for the implant, and the wireless power transmitter is intended for use outside the body.

3. The apparatus as recited in claim 1, wherein the thermistor is disposed in the apparatus in temperature-measuring proximity of the Zener diode.

4. The apparatus as recited in claim 1, wherein the rectifier is electrically connected to a resonator that is configured to generate current when exposed to a field generated by the wireless power transmitter.

5. The apparatus as recited in claim 1, wherein the remedial overvoltage protection actions include activating a circuit that reduces a voltage applied to the Zener diode.

6. The apparatus as recited in claim 1, further comprising an overvoltage protection circuit, and wherein the remedial overvoltage protection actions include triggering the overvoltage protection circuit to:
short one or more input terminals of the rectification circuit to ground; and
responsive to detection that the measured temperature has decreased below an operational temperature threshold, discontinue shorting the one or more input terminals to ground.

7. The apparatus as recited in claim 1, further comprising a communication component, and wherein the remedial overvoltage protection actions include causing the communication component to communicate a notification indicative of an overvoltage protection condition to the wireless power transmitter.

8. The apparatus as recited in claim 7, wherein the communication component is configured to communicate with the wireless power transmitter using at least one of an in-band communication technique or an out-of-band communication technique.

9. The apparatus as recited in claim 7, wherein the notification is configured according to a communication standard for communicating notifications indicative of overvoltage protection conditions from implants to wireless power transmitters.

10. The apparatus as recited in claim 1, wherein the wireless power transmitter is configured to wirelessly transmit the power to multiple implants implanted in a body.

11. The apparatus as recited in claim 1, wherein the apparatus is a power receiving unit that is incorporated in an implant intended for use in a body.

12. The apparatus as recited in claim 1, wherein the Zener diode is configured to clamp the DC power at the predefined voltage utilizing a reverse voltage of the Zener diode.

13. The apparatus as recited in claim 1, further comprising one or more power-conditioning components disposed in the rectification circuit to condition at least one of the AC or DC power, including at least one of:
a capacitor;
a Schottky diode; or
a low-dropout regulator.

14. A method for providing overvoltage protection for an electronic device that receives power wirelessly from a wireless power transmitter, the method comprising:
receiving alternating current (AC) power at a power receiving unit's rectification circuit, the AC power generated based on wireless power received from the wireless power transmitter;
rectifying the AC power to direct current (DC) power with a rectifier of the rectification circuit;
clamping a voltage of the DC power at a predefined voltage using a Zener diode of the rectification circuit, the clamped DC power that is output at an output terminal of the rectification circuit being usable for operation of the electronic device;
determining that a measured temperature of the Zener diode exceeds a threshold temperature; and
initiating one or more remedial overvoltage protection actions when the measured temperature exceeds the threshold temperature.

15. The method as recited in claim 14, wherein the electronic device is an implant intended for use in a body and the overvoltage protection protects the implant.

16. The method as recited in claim 14, further comprising ceasing the one or more remedial overvoltage protection actions when the measured temperature of the Zener diode falls below an operational temperature threshold that indicates the Zener diode can be safely used to clamp the voltage of the DC power.

17. The method as recited in claim 14, further comprising measuring a temperature of the Zener diode with a thermistor in temperature-measuring proximity of the Zener diode.

18. The method as recited in claim 14, wherein the remedial overvoltage protection actions include triggering an overvoltage protection circuit to reduce a voltage applied to the Zener diode.

19. The method as recited in claim 14, wherein the remedial overvoltage protection actions include notifying the wireless power transmitter of an overvoltage protection condition, the notifying enabling the wireless power transmitter to adjust wireless power transmitted to remedy the overvoltage protection condition.

20. The method as recited in claim 14, further comprising configuring a notification indicative of an overvoltage protection condition for communication to the wireless power transmitter according to a standard that specifies how implants and wireless transmitters are to handle overvoltage protection conditions.

21. The method as recited in claim 14, wherein the power receiving unit is surgically implanted in a body.

22. The method as recited in claim 14, further comprising operating the electronic device using the clamped DC power that is output at the output terminal.

23. An apparatus comprising:
biomedical-implant circuitry configured to provide biomedical functionality for a body; and
a power receiving unit configured to receive from a wireless power transmitter power that the biomedical-implant circuitry uses to provide the biomedical functionality, the power receiving unit further configured to provide overvoltage protection for the biomedical-implant circuitry, the power receiving unit including:
a rectifier disposed in a rectification circuit of the power receiving unit, the rectifier configured to rectify alternating current (AC) power received at the rectification circuit to direct current (DC) power;
a Zener diode disposed in the rectification circuit between the rectifier and an output terminal of the rectification circuit, the Zener diode configured to clamp the DC power at a predefined voltage, the clamped DC power output at the output terminal being usable by the biomedical-implant circuitry to provide the biomedical functionality; and
a thermistor configured to measure a temperature of the Zener diode, the measured temperature correlating to voltage at the Zener diode due to clamping and triggering one or more remedial overvoltage protection actions when the measured temperature exceeds a threshold temperature.

24. The apparatus as recited in claim 23, wherein the thermistor is disposed in the apparatus in temperature-measuring proximity of the Zener diode.

25. The apparatus as recited in claim 23, further comprising an overvoltage protection circuit that is configured to reduce a voltage applied to the Zener diode, the overvoltage protection circuit being activated to reduce the voltage applied to the Zener diode as one of the remedial overvoltage protection actions.

26. The apparatus as recited in claim 23, further comprising a communication component, and wherein the remedial overvoltage protection actions include causing the communication component to communicate a notification indicative of an overvoltage protection condition to the wireless power transmitter.

27. The apparatus as recited in claim 23, wherein the biomedical functionality comprises at least one of:
releasing metered doses of medication;
stimulating tissue of the body; or
monitoring one or more biochemical conditions.

28. An apparatus for providing overvoltage protection in connection with receiving power wirelessly from a wireless power transmitter, the apparatus comprising:
a rectification means for rectifying alternating current (AC) power received to direct current (DC) power;
a voltage-clamping means for clamping the DC power at a predefined voltage, the clamped DC power output from the rectification means being usable by the apparatus; and
a temperature-measuring means for measuring a temperature of the voltage-clamping means, the measured temperature correlating to voltage at the voltage-clamping means and being used to detect an overvoltage protection condition.

29. The apparatus as recited in claim 28, wherein the temperature-measuring means is disposed in temperature-measuring proximity to the voltage-clamping means.

30. The apparatus as recited in claim 28, further comprising one or more overvoltage protection remedying means for effectuating one or more remedial overvoltage protection actions responsive to detection of the overvoltage protection condition, the remedial overvoltage protection actions including locally effectuated overvoltage protection actions and remotely effectuated overvoltage protection actions.
